(19) United States
(12) Patent Application Publication
(10) Pub. No.: US 2006/0171983 A1
(43) Pub. Date: Aug. 3, 2006

(54) Use of Vitamin D receptor activators or Vitamin D analogs to treat cardiovascular disease

(76) Inventors: Jin Tian, Waukegan, IL (US); Joel Z. Melnick, Wilmette, IL (US); E. Scott Toner, Vernon Hills, IL (US); Jinshyun Ruth Wu-Wong, Libertyville, IL (US); David H. Ostrow, Lake Zurich, IL (US); Laura A. Williams, Gurnee, IL (US); Eugene Sun, Libertyville, IL (US); Dheerendra R. Kommala, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/176,103

(22) Filed: Jul. 7, 2005

Related U.S. Application Data
(63) Continuation-in-part of application No. 11/002,934, filed on Dec. 2, 2004.
Application No. 11/002,934 is a continuation-in-part of application No. 10/903,039, filed on Jul. 29, 2004.
(60) Provisional application No. 60/530,842, filed on Dec. 18, 2003.
(60) Provisional application No. 60/491,088, filed on Jul. 30, 2003.

(51) Int. Cl. A61K 31/59; A61L 15/16; A61F 13/02

(52) U.S. Cl. 424/422; 514/167; 424/448

(57) ABSTRACT

Disclosed are pharmaceutical compositions containing Vitamin D receptor activators or Vitamin D analogs to treat, prevent or inhibit vascular disease among other conditions. The pharmaceutical compositions may also include ACE inhibitors or other agents. Also disclosed are methods of reducing PAI-1 expression by administering effective amounts of Vitamin D receptor activators or Vitamin D analogs to a mammal in need thereof. Additionally disclosed are methods of preventing, inhibiting or treating thrombosis in a mammal in need of such prevention, inhibition or treatment comprising administering effective amounts of Vitamin D receptor activators or Vitamin D analogs to the mammal.

### CROSS REFERENCE TO RELATED APPLICATIONS

The subject application is a Continuation-In-Part of and claims priority to pending U.S. patent application Ser. No. 11/002,934, filed on Dec. 2, 2004, which claims priority to and is a Continuation-In-Part of 1) pending U.S. patent application Ser. No. 10/903,039, filed on Jul. 29, 2004 and also claims priority to 2) U.S. Provisional Application No. 60/530,842, filed on Dec. 18, 2003, now abandoned. U.S. patent application Ser. No. 10/903,039, filed Jul. 29, 2004, claims priority to U.S. Provisional Application No. 60/491,088, filed on Jul. 30, 2003, now abandoned. All of the cited applications are hereby incorporated in their entirety by reference.

### FIELD OF THE INVENTION

The present invention relates to the use of a Vitamin D receptor activator (VDRA), preferably paricalcitol, or a Vitamin D analog, to treat and prevent cardiovascular disease, including cerebrovascular and peripheral vascular diseases, especially heart failure, cardiomyopathy, atherosclerosis, myocardial infarction, and cerebrovascular accidents.

### BACKGROUND OF THE INVENTION

Complications of cardiovascular diseases (CVD) due to atherosclerosis and cardiomyopathy are the most common cause of death in Western societies. Hypertension and hyperlipidemia in particular are major cardiac risk factors. Certain medications that treat hypertension (e.g., angiotensin converting enzyme inhibitors (ACEIs)) and abnormal lipid levels have been proven to reduce cardiovascular mortality significantly in high-risk populations such as hemodialysis patients. However, several factors, including adverse side effects, limit the utility of existing medications for preventing progression of cardiovascular disease or otherwise render these medications inadequate for treatment of CVD, particularly critical for high-risk populations.

The biological effects of VDRAs are mediated by the vitamin D receptor (VDR), a member of the superfamily of nuclear hormone receptors. One mechanism by which the VDR is believed to mediate biological effects is through activation of a transcription factor that binds to specific DNA sequence elements in vitamin D responsive genes and ultimately influences the rate of RNA polymerase II mediated transcription. VDRs are present in most human cell types, especially in the cardiovascular system and immune system.

Several lines of evidence support the idea that vitamin D plays an important role in the regulation of cardiovascular physiology as described in FIG. 1. Vitamin D has the potential to prevent atherosclerosis and vascular calcification through its effects on the immune system to down-regulate inflammatory pathways and to restore the normal expression of inhibitors of vascular calcification. Vitamin D also effects cell proliferation. Low vitamin D levels were associated with congestive heart failure. Vitamin D has direct effects to antagonize endothelin-1 induced cardiomyocyte hypertrophy. Finally, VDRAs down-regulate RAAS by inhibiting renin synthesis. Thus, treatment with VDRAs/vitamin D analogs may prevent or treat cardiovascular disease by affecting one or all of the pathways in FIG. 1.

However, in vitro and animal data have suggested that VDRAs and/or Vitamin D analogs can damage the heart in uremic patients, for example, by causing vascular calcification, myocardial infarction, heart failure, cardiomyopathy and cerebrovascular accidents. Therefore, the medical community does not endorse use of these compounds as a therapy for cardiovascular disease and recommends the limitation of their use.

### SUMMARY OF THE INVENTION

The present invention is directed to methods for preventing, treating and delaying progression of vascular diseases, including cardiovascular, cerebrovascular and peripheral vascular diseases, especially heart failure, cardiomyopathy, atherosclerosis, myocardial infarction, and cerebrovascular accidents and pharmaceutical compositions useful therefor.

According to one embodiment, the present invention relates to VDRAs or Vitamin D analogs (referred to herein as “VDRA/Vitamin D analog”)-containing compositions for preventing, treating and delaying progression of vascular disease

According to some aspects of the present invention, Vitamin D receptor activator (VDRA) compounds can be used. VDRAs include paricalcitol, calcitriol, 22-oxa-1-alpha,25-dihydroxyvitaminD2, MC-903 (calcipotriol), 16-ene-23-yne-1alpha, 25-dihydroxyvitamin D3, and 24-difluoro-26,27-dimethyl-16-ene-1alpha, 25-dihydroxyvitamin D3 (described in greater detail by DeLuca, et al., in PNAS, 2004, vol. 101, No. 18, p. 6900-6904, incorporated herein by reference), compounds listed in Table 1 of Physiol. Rev. October 1998, Vol. 78, No. 4, p 1193-1231, incorporated herein by reference in its entirety, and the so-called Gemini compounds (described in greater detail by Maehr, et al. in J. Steroid Biochem. Mole. Biol. 89-90, 2004, 35-38, incorporated herein by reference), EB-1089 (a LEO Pharmaceuticals compound), and ED-71 (a Roche compound). Paricalcitol is especially preferred since it is a selective VDRA. Paricalcitol is commercially available from Abbott Laboratories (North Chicago, Ill., under the tradename ZEMPLAR).

According to other aspects of the present invention, the Vitamin D analog can be doxercalciferol or alfacalcidol.

According to some embodiments, especially preferred compositions of the present invention also include one or more of the following agents: an angiotensin converting enzyme inhibitor (ACEI) or an angiotensin II receptor 1(AT1) blocker or an aldosterone blocker (ARB). Compositions according to the present invention can also include other agents used to treat or prevent cardiovascular disease, such as beta blockers, calcium channel blockers, antilipemic agents, antihypertensive agents and antiinflammatory agents, including aspirin.

According to some aspects of the invention, pharmaceutical compositions can be administered through a sustained (or continuous) delivery system. The present invention also contemplates other modes of administration, including but not limited to oral, injectable and transdermal.

The present invention also includes a method of treating, inhibiting or preventing thrombosis in a mammal in need of such treatment, inhibition or prevention, comprising the step of administering to the mammal an effective amount of a Vitamin D receptor activator or Vitamin D analog. The Vitamin D receptor activator may be, for example, paricalcitol or calcitriol, and the Vitamin D analog may be, for example, doxercalciferol or alfacalcidol.

All patents and publications referred to herein are hereby incorporated in their entirety by reference.

### BRIEF DESCRIPTION OF THE FIGURES

FIG. 1 schematically represents the role of Vitamin D in deregulation of various inflammatory factors associated with atherosclerosis and its association with cardiomyocyte remodeling.

FIG. 2 presents bar graphs comparing median hospitalizations per year and hospital days per year for paricalcitol, calcitriol and no D therapy.

FIG. 3 presents bar graphs comparing results of regression analysis showing treatment with paricalcitol was associated with fewer hospitalizations and hospital days per year compared to no D.

FIG. 4 illustrates a Northern blot which evidences that paricalcitol treatment of As4.1-hVDR cells dose-dependently inhibits renin mRNA expression.

FIG. 5 illustrates the results of a renin promoter-luciferase assay used to examine the activity of paricalcitol to suppress renin gene transcription.

FIG. 6 illustrates the effect of paricalcitol and calcitriol on PAI-1 in primary culture of human coronary artery smooth muscle cells.

FIG. 7 illustrates the effect of vitamin D analogues on expression of the NPR-A gene promoter. VD3 represents 1,25 dihydroxyvitamin D (all results are normalized for co-transfected CMV Renilla luciferase expression).

FIG. 8 shows the effect of vitamin D analogues on ANP-stimulated cyclic GMP accumulation where ANP-dependent cGMP generation was used as a surrogate for ANP activity.

FIG. 9 shows the effect of vitamin D analogues on mutant (VDRE-deleted) NPR-A gene promoter in neonatal rat aortic smooth muscle cells; results are normalized for Renilla luciferase expression. Results suggest that all tested compounds induce ANP through the vitamin D response element.

FIG. 10 shows the effect of vitamin D analogues on basal vs. endothelin (10⁻⁷ M) stimulated hBNP gene promoter activity using transfected cardiac myocytes that were cultured in serum free medium.

FIG. 11 shows the effect of vitamin D analogues on basal and endothelin (10⁻⁷ M) stimulated hBNP gene promoter activity using transfected cardiac myocytes cultured in 0.2% fetal bovine serum. All cells were co-transfected with expression vectors directing expression of hVDR and hRXR.

FIG. 12 shows the effect of vitamin D analogues on basal and endothelin (10⁻⁷ M) stimulated Cdk2 activity in neonatal rat aortic smooth muscle cells.

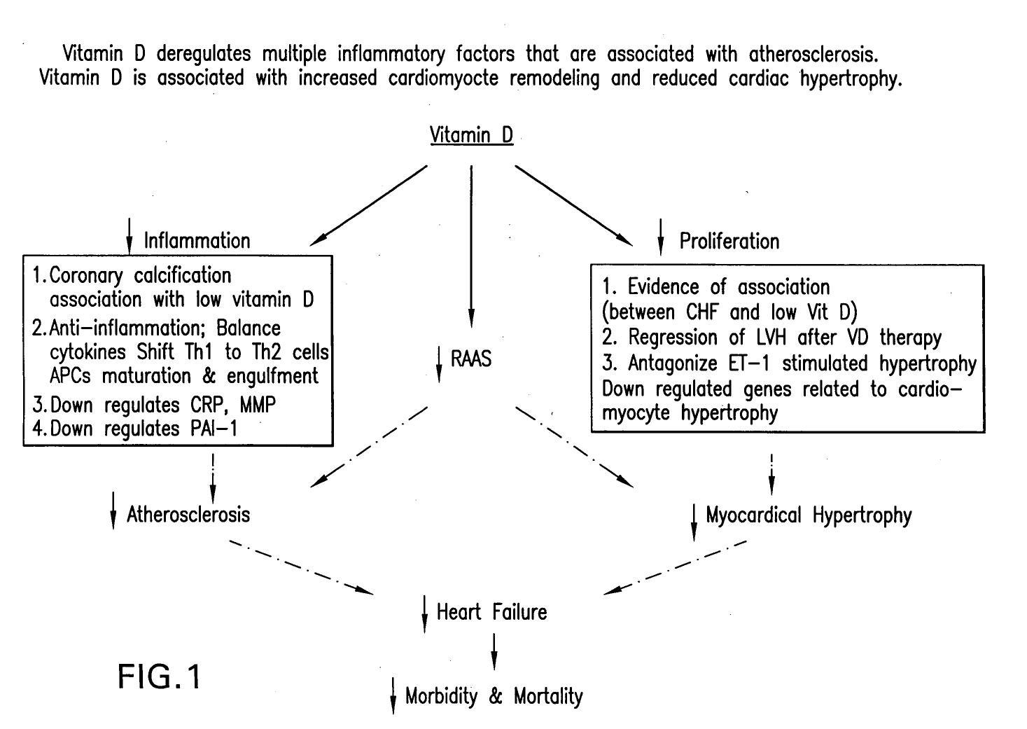

### DESCRIPTION OF THE EMBODIMENTS OF THE INVENTION

The present invention is generally directed to compositions containing a VDRA/Vitamin D analog to treat or prevent cardiovascular diseases (CVD), including cardiomyopathy, coronary arterial, cerebrovascular and peripheral vascular diseases. The present invention also relates to methods of treating CVD by administering to a patient a pharmaceutical composition, which may be a sustained release formulation, containing a therapeutically effective amount of a VDRA/Vitamin D analog.

Treatment of patients with CVD by administration of a therapeutically effective amount of a VDRA/vitamin D analog-containing composition is expected to be advantageous for effective reduction of renin expression, decreased inflammation and improved cardiac function directly through the therapeutic action of the VDRA/Vitamin D analog on cardiac tissue. In contrast, conventional treatments based on administration of an ACEI (i.e., without a VDRA/Vitamin D analog) for example, only reduce angiotensin (II), but do not reduce renin levels or act on Vitamin D receptors in the heart, vasculature and immune system itself. Administration of ACEI may not be an attractive long term treatment due to adverse consequences.

According to some aspects of the present invention, the inventive compositions contain a VDRA/Vitamin D analog and at least one of the following agents: an ACE inhibitor, an angiotensin (II) receptor blocker (ARB) and aldosterone blocker in therapeutically effective amounts to inhibit renin production or inhibit activation of the renin-angiotensin-aldosterone system. Preferred compositions contain paricalcitol with at least one of these other agents. Such combinations can avoid ACE inhibition escape and aldosterone escape with subsequent increase in angiotensin (II) and aldosterone generation

Suitable ACE inhibitors, ARB and aldosterone blockers are commercially available. Suitable ACE inhibitors include, but are not limited to: captopril (commercially available under the tradename CAPOTEN from Mylan), enalapril (commercially available under the tradename VASOTEC from Merck), fosinapril (commercially available under the tradename MONOPRIL from Bristol Myers Squibb), benzapril (commercially available under the tradename LOTENSIN from Novartis Pharmaceuticals), moexipril (commercially available under the tradename UNIVASC from Schwarz Pharma), perindopril (commercially available under the tradename ACEON from Solvay), quinapril (commercially available under the tradename ACCUPRIL from Parke-Davis), ramipril (commercially available under the tradename ALTACE from Monarch), trandolapril (commercially available under the tradename MAVIK from Abbott Laboratories of North Chicago, Ill.), lisinopril (commercially available under the tradenames PRINIVIL from and ZESTRIL from Astra Zeneca).

Suitable angiotensin receptor blocking agents include, but are not limited to: losartan (commercially available as COZAAR from Merck), irbesartan (commercially available as AVAPRO from Bristol Myers Squibb and Sanofi), candesartan (commercially available as ATACAND from Astra Zeneca), eprosartan (commercially available as TEVETEN from Biovail Corporation of Canada), telmisartan (commercially available as MICARDIS from Boehringer Ingelheim) and valsartan (commercially available as DIOVAN from Novartis).

Suitable aldosterone blockers include, but are not limited to: eplerenone (commercially available under the tradename INSPRA from Pharmacia), spironolactone (commercially available under the tradenames Aldactone, Adultmin, Aldopur, Aldospirone, Almatol, Berlactone, Diatensec, Diram, Esekon, Hypazon, Idrolattone, Merabis, Novospiroton, Osiren, Osyrol, Pirolacton, Resacton, Sincomen, Spiractin, Spiroctan, Spirolacton, Spirolang, Spironex, Spirotone, Tevaspirone, Verospiron, Xenalon Lactabs, Youlactone).

Additional components, e.g., physiologically acceptable carriers, solvents, binders, antioxidants, colorants, substrates can be used as necessary or desired.

Preferred treatment or preventative regimens for patients with CVD according to the present invention would administer therapeutically effective VDRA/Vitamin D analog-containing compositions according to the invention for a sufficient period to effect sustained or continuous delivery. As used herein, a “therapeutically effective dose” is a dose which in susceptible subjects is sufficient to prevent progression or cause regression of CVD or which is capable of relieving the symptoms caused by CVD.

An exemplary dosing regimen would provide the equivalent of about 0.5 micrograms of calcitriol per day or at least about 1 microgram calcitriol by injection three times weekly. For paricalcitol, a suitable dosing regimen would provide the equivalent of about 2 micrograms paricalcitol daily or at least about 4 micrograms paricalcitol three times weekly administered as a bolus. Suitable dosing regimens for other VDRA/Vitamin D analogs, e.g., doxercalciferol, can be determined straightforwardly by those skilled in the art based on the therapeutic efficacy of the VDRA/Vitamin D analog to be administered.

Since ACEI, ARB and aldosterone inhibitors have different efficacies and affect the body through different pathways than Vitamin D does, compositions according to the present invention can incorporate an ACEI, ARB or aldosterone inhibitor to be administered according to conventional dosing regimens, which are well known and readily available to those skilled in the art.

The invention also contemplates continuous or sustained drug delivery forms containing the selected VDRA/Vitamin D analog, and an ACEI and/or an ARB and/or an aldosterone blocker. Suitable delivery forms include, but are not limited to, tablets or capsules for oral administration, injections, transdermal patches for topical administration (e.g., drug to be delivered is mixed with polymer matrix adhered to or absorbed on a support or backing substrate, e.g., ethylcellulose), depots (e.g., injectable microspheres containing the desired bioactive compounds) and implants. Techniques for making these drug delivery forms are well known to those skilled in the art.

Further, it should also be noted that CKD patients undergoing hemodialysis often require the formation of an arteriovenous (A-V) fistula for hemodialysis (HD). The autogenous A-V fistula has long been proven to be the most durable access for HD. Primary failure of vascular access is mainly related to thrombosis. The pathophysiology underlying stenosis formation is turbulence of blood flow, which activates platelets and endothelial cells. The final trigger causing thrombosis is a critical reduction of fistula blood flow. In this context, a particular role has been postulated for platelet-derived growth factor (PDGF)

Based upon the data presented in Example 5 below, it can be concluded that there is a statistically significant association with Zemplar therapy and fewer vascular access changes. Thus, Zemplar may have a beneficial effect through its action on endothelial cells, platelets and PDGF which are responsible for thrombosis. Future studies should clarify the mechanism of the proposed effect, understand if it extends beyond AV fistulas to grafts, dose-time dependency and the association with CaxP Product.

The present invention may be illustrated by the used of the following, non-limiting examples:

### EXAMPLE 1

### Decreased Morbidity and Mortality Associated with Vitamin D Therapy

The leading cause of mortality and morbidity in patients receiving chronic hemodialysis related to cardiovascular disease. Prevalence of CVD can be found in at least 75% of patients who initiate hemodialysis therapy.

An observational cohort study examining hemodialysis patients who started vitamin D therapy with paricalcitol experienced fewer hospitalizations related to cardiovascular events and non-infectious inflammations, compared with patients treated with calcitriol (Paricalcitol-treated patients experience improved hospitalization outcomes compared with calcitriol-treated patients in real-world clinical settings, D. G. Dobrez, et al. Nephrol Dialysis Transplant 2004 19:1174).

This study was expanded to include patients who received no Vitamin D receptor activator treatment. [“Improved hospitalization outcomes in hemodialysis patients treated with paricalcitol.” J. Melnick, et al., abstract book from World Congress of Nephrology, Jun. 8-12, 2003, Berlin. Page 148] revealed that paricalcitol treatment was associated with improved hospitalization outcomes in hemodialysis (HD) patients who were treated with paricalcitol or with calcitriol compared to patients who did not receive any vitamin D treatment.

As shown in FIG. 2, evaluation of hospitalization endpoints revealed the median number of hospitalizations in a year for patients receiving a VDRA (either paricalcitol (“Par”) or calcitriol (“Cal”)) was lower than for patients who received no Vitamin D (“No D”). Notably, hospitalizations were fewer for patients treated with paricalcitol (1.5) than for those treated with calcitriol (2.2). In addition, the median number of days spent in the hospital was lower for patients receiving a VDRA (especially paricalcitol) compared to patients who received no Vitamin D (2.6). The number of hospital days was again lowest for paricalcitol (5.2) compared to calcitriol (10.6) and no Vitamin D (14.7).

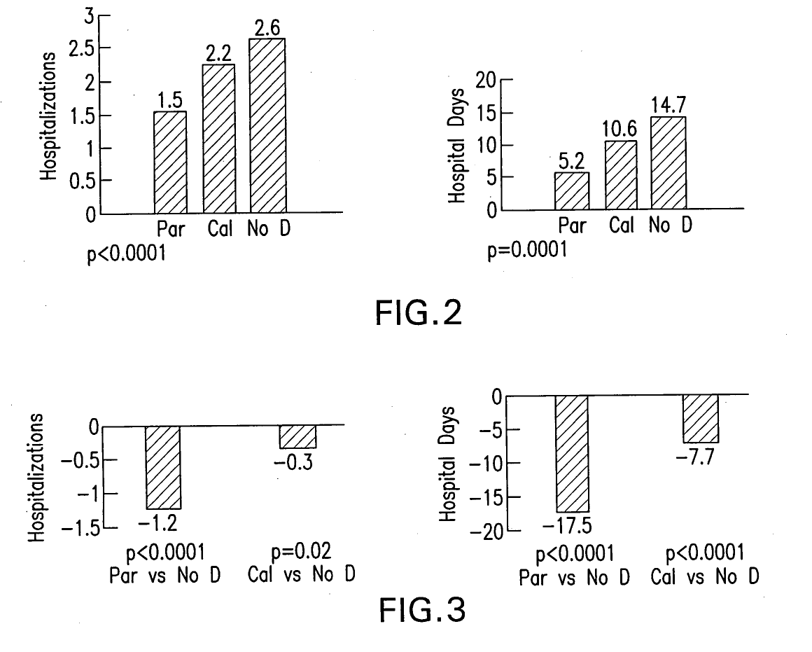

FIG. 3 presents multivariate results for the hospitalizations and hospital days per year. Regression analysis of this data revealed receiving calcitriol was associated with 7.7 fewer hospitalization days compared to the No Vitamin D group, even though there was no statistical difference in the number of hospitalizations. However, treatment with paricalcitol was associated with 1.2 fewer hospitalizations and 17.5 fewer hospital days compared to the No Vitamin D group.

### EXAMPLE 2

### Activity of Paricalcitol to Suppress Renin Expression

Recently, it has been found that 1,25-dihydroxyvitamin D functions as a negative regulator of renin biosynthesis in vitro and in in vivo studies. Calcitriol is able to inhibit renin gene expression, which provides a molecular basis to explore the use of vitamin D and vitamin D analogs as new renin inhibitor to regulate rennin-angiotensin-aldosterone system (RAAS).

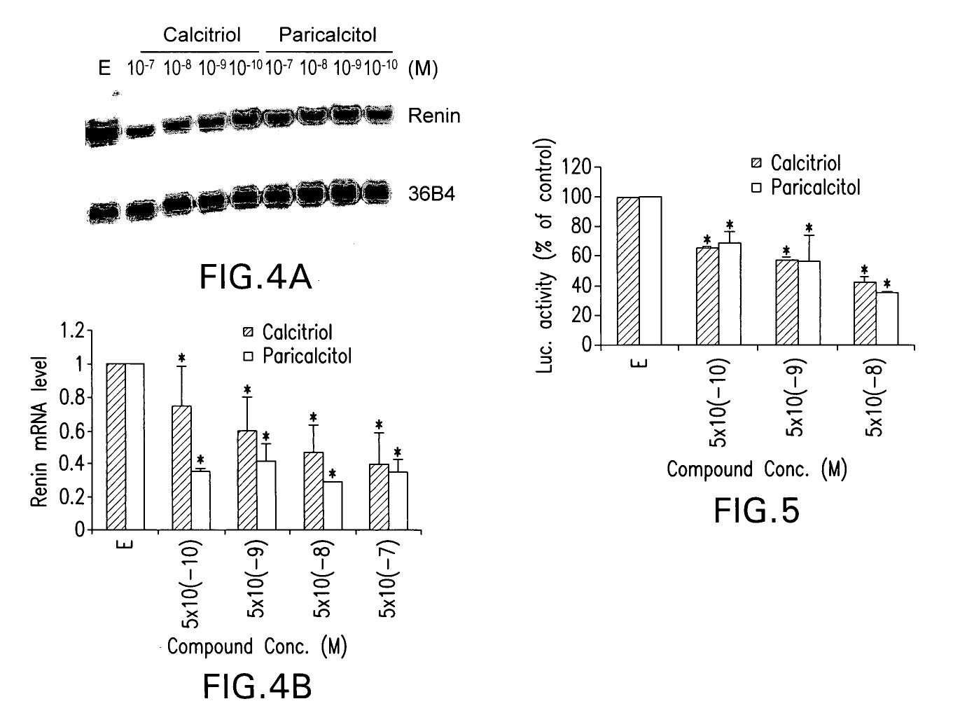

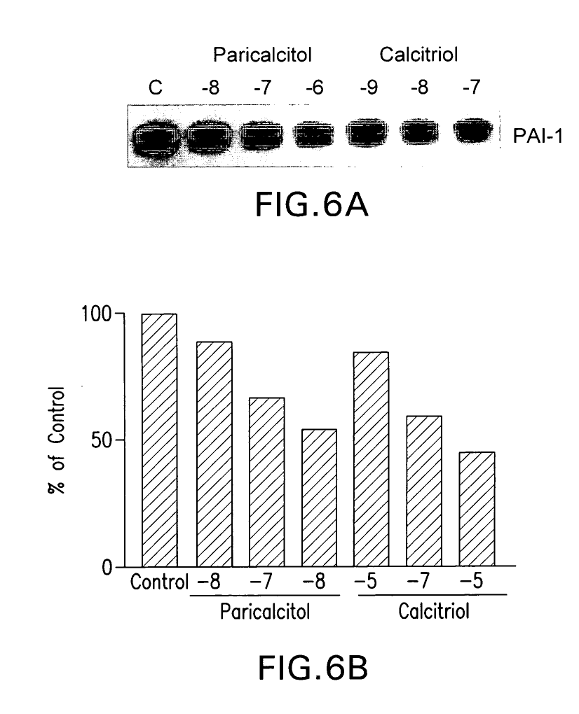

Using an in vitro cell culture system, the activity of paricalcitol to suppress renin gene expression was examined using previously published techniques (1,25-Dihydroxyvitamin D₃ is a negative endocrine regulator of the renin-angiotensin system, J. Clin. Invest., July 2002). As shown in FIG. 4, by Northern blot analysis, paricalcitol treatment of As4.1-hVDR cells dose-dependently inhibits renin mRNA expression. In fact, its renin-inhibiting activity appears a bit more potent than calcitriol (FIGS. 4A and B). This inhibitory effect is confirmed by renin promoter-luciferase reporter assays, which examine the activity of paricalcitol to suppress renin gene transcription. In these assays, paricalcitol appears at least as potent as calcitriol to suppressing the activity of the renin gene promoter (FIG. 6).

This data supports the utility of a VDRA/Vitamin D analog to regulate the renin-angiotensin-aldosterone system and its criticality in CVD development and delay in progression of cardiovascular disease.

### EXAMPLE 3

### Effect of VDR Activators on PAI-1

The effect of paricalcitol and calcitriol on PAI-1 in primary culture of human coronary artery smooth muscle cells was investigated. (See FIG. 6.) PAI-1 (plasminogen activator inhibitor type-1) is one of the risk markers for coronary heart disease, and is enhanced in atherosclerotic plague and colocalized with macrophages.

Human coronary artery smooth muscle cells were incubated with paricalcitol or calcitriol at the indicated concentration for 24 hr at 37° C. Samples were solubilized in SDS-PAGE sample buffer, and the protein content in each sample was determined by the Bio-Rad dye-binding protein assay. Samples were resolved by SDS-PAGE using a 4-12% gel, and proteins were electrophoretically transferred to PVDF membrane for Western blotting. The membrane was blotted for 1 h at 25° C. with 5% nonfat dry milk in PBS-T and then incubated with a mouse anti-PAI-1 monoclonal antibody in PBS-T overnight at 4° C. The membrane was washed with PBS-T and incubated with a horseradish peroxidase-labeled anti-rabbit antibody for 1 h at 25° C. The membrane was then incubated with detection reagent (SuperSignal WestPico). The specific bands were visualized by exposing the paper to Kodak BioMax films.

FIG. 6 shows the results from Western blot using an anti-PAI-1 antibody. Two observations may be noted in these studies: (1) 100% inhibition of growth was never achieved even at 1 μM of any of the test compound. Confocal microscopy studies confirm that, although these drugs are potent in inducing the translocation of VDR from cytoplasm to nucleus, not all cells respond to VDRAs even after 2 h of exposure, which may explain the <100% inhibition. (2) Although paricalcitol is known to be less potent than calcitriol in the clinical studies, it exhibits similar potency to calcitriol in this assay. By checking the effect of drugs on the expression of 24(OH)ase, it was found that paricalcitol is less potent than calcitriol on stimulating the expression of 24(OH)ase, which may partially explain the higher potency of paricalcitol in this assay. These results show that paricalcitol and calcitriol are equally potent in reducing the PAI level in human coronary artery smooth muscle cells. Paricalcitol is usually dosed approximately 4 fold higher than calcitriol in the clinical situation, which may translate into a 4-fold higher potency in regulating the function of smooth muscle cells.

### EXAMPLE 4

### Effect of Paricalcitol in In Vitro Models Using Myocardial or Vascular Smooth Muscle Cells in Culture

Experimentally induced vitamin D deficiency is associated with cardiac hypertrophy and hypertension in otherwise normal adult Sprague-Dawley rats (Weishaar et al., Am. J. Physiol. 1990 January; 258 (1 Pt 1):E134-42). Cardiac hypertrophy is also seen in the VDR −/− mouse (Li et al., J. Clin. Invest. 2002 July; 110 (2):229-38), although this occurs in the setting of a 10-15 mm Hg elevation in systolic blood pressure implying that the hypertrophy may, as least in part, reflect increased ventricular overload. Vitamin D has been shown to inhibit endothelin (ET)-induced hypertrophy of neonatal rat cardiac myocytes in culture (Wu et al., J. Clin. Invest. 1996 Apr. 1; 97(7):1577-88 and Li et al., J. Biol. Chem. 1994 Feb. 18; 269(7):4934-9). This is associated with a reduction in expression of the ANP, BNP and α skeletal actin genes and suppression of the human ANP and BNP gene promoters (Wu et al., Am. J. Physiol. 1995 June; 268 (6 Pt 1):E1108-13.

In the present study, an examination was made as to whether paricalcitol possesses similar effects (vs. the native hormone) in several in vitro models using myocardial or vascular smooth muscle cells in culture.

Effect of VDRA/Vitamin D Analogs on NPR-A Gene Promoter Activity.

Neonatal RASM cells were transfected with −1575 NPR-A LUC (0.5 μg) by electroporation. Cells were co-transfected with a constitutively active CMV-Renilla luciferase reporter (0.25 μg) to control for differences in transfection efficiency. 24 hrs post-transfection, cells were treated with the vitamin D analogues, or vehicle, as indicated. The incubation was continued for 48 hrs at which point cells were harvested, lysates were generated and luciferase (firefly and Renilla) measurements were made.

Effect of VDRA/Vitamin D Analogs on NPR-A Activity

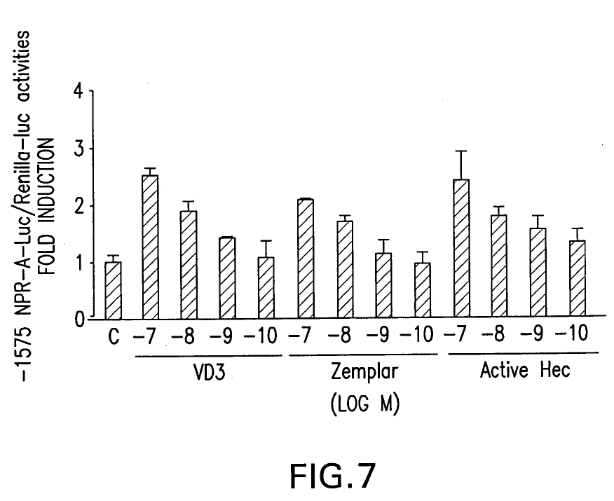

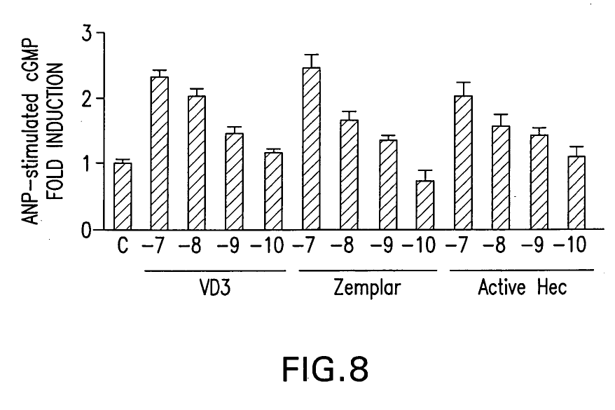

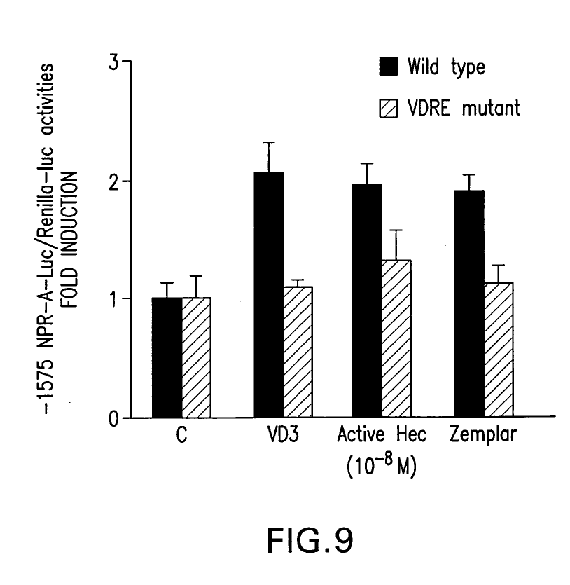

Cells were preincubated for 48 hrs in 1,25 dihydroxyvitamin D (VD), paricalcitol, HECTEROL® (calcitriol) or the activated form of HECTEROL (calcitriol). At that point medium was changed, the non-selective phosphodiesterase inhibitor IBMX (10⁻⁴ M) was added, and the incubation was continued for 10 min at 37 C. ANP (10⁻⁷ M) was then added to each culture and the incubation extended an additional 10 minutes. Medium was then aspirated, cells were lysed with TCA and soluble extracts subjected to ether extraction, neutralization and radioimmunoassay for cGMP levels. All cGMP levels presented here are normalized per μg of soluble protein present in the extract. Results are shown in FIGS. 7, 8 and 9.

Effect of Vitamin D Analogues on hBNP Gene Promoter Activity.

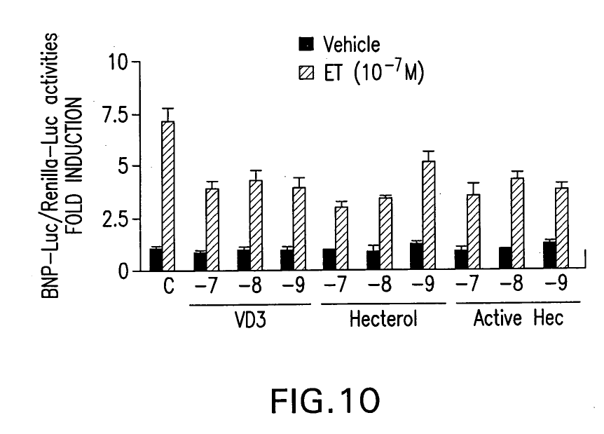

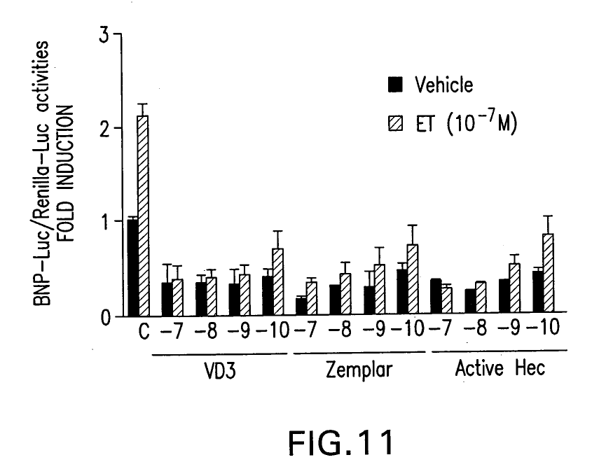

Neonatal rat ventricular myocytes were transfected with −1595 hBNP LUC (0.25 μg) by electroporation as described previously. Co-transfected CMV-Renilla luciferase (0.25 μg) was used to normalize samples for differences in transfection efficiency, as described above. In selected cases, expression vectors for the human vitamin D receptor (hVDR) (0.3 μg) and human retinoid X receptor (hRXR) (0.3 μg) were co-transfected with the BNP luciferase reporter. Where indicated samples were treated with endothelin (10⁻⁷ M) or one of the vitamin D analogues. Results are shown in FIGS. 10 and 11.

Measurement of Cdk2 Activity.

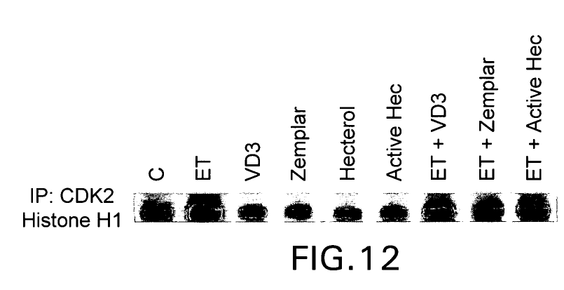

Cells were treated with vehicle or the vitamin D analogues for the intervals indicated. Cells were lysed with lysis buffer and 100 μg of supernatant protein was incubated with 1 μg of anti-Cdk2 antibody and 10 μl of protein G-Sepharose for 1-2 hrs at 4 C. Immune complex kinase assays were carried out as described previously using the immunoprecipitates generated above together with 2 μg of histone 1 and γ-³²P-ATP in kinase buffer. Reaction products were separated on denaturing SDS-polyacrylamide gels that were then dried and exposed to X-ray film. Results are shown in FIG. 12.

The current study indicates that VDRAs possess functional activity in the cardiovascular system that is similar, both qualitatively and quantitatively, to that previously demonstrated for the native hormone, 1,25 dihydroxyvitamin D. Specifically, the major findings of this study indicate that VDRAs: 1) increase activity of the type A natriuretic peptide receptor (NPR-A) in neonatal rat aortic smooth muscle cells, 2) increase NPR-A gene promoter activity in the same cells through a vitamin D response element, 3) suppress ET-dependent stimulation of the BNP gene promoter in cultured neonatal rat ventricular myocytes, 4) inhibit endothelin-dependent stimulation of ³H-thymidine incorporation into DNA and Cdk2 activity in adult rat aortic smooth muscle cells. Collectively, these data suggest that paricalcitol, like 1,25 dihydroxyvitamin D, may possess cardio-protective effects that control hypertrophy of cardiac myocytes in the myocardial wall and vasculo-protective effects that both limit cell proliferation in the remodeling vascular wall and increase the expression/activity of the anti-proliferative, vasorelaxant natriuretic peptide/NPR system in the vasculature.

### EXAMPLE 5

### Vascular Access Changes in Subjects Treated with Zemplar

Methods: A historical cohort of 2112 adult patients new to HD, with an AV fistula as the initial primary vascular access were followed over a 35-month period (January 1999 thru November 2001) using a dialysis provider database. Patients were treated with Zemplar or no vitamin D therapy; patients receiving Zemplar therapy received at least 10 doses and remained on the same therapy. Descriptive summary statistics were used to summarize baseline characteristics and the total number of vascular access changes per year between treatment modalities. In addition, regression models were used to evaluate the association between Zemplar or no vitamin D therapy and the total number of vascular access changes per year.

Results: The data set contained 577 patients treated with Zemplar and 1535 patients who received no vitamin D therapy. The total number of vascular access changes averaged 0.6 changes per year in Zemplar patients and 0.9 changes per year in no D Patients (p=0.0034). Negative binomial regression was performed to control for baseline covariates; this revealed that the No D group were associated with 28% more vascular access changes than Zemplar patients (p=0.038).

### EXAMPLE 6

### Effects of Genetic 1α,25-(OH)₂D₃ Deficiency on Blood Pressure and Cardiovascular Phenotype in CYP27B1 (1-α-Hydroxylase) Knock-Out Mice

It has been demonstrated that 1α,25-(OH)₂D₃ (calcitriol) is a negative regulator of the renin-angiotensin system. Thus, it was thought that perhaps mice lacking CYP27B1 (1-alpha hydroxylase), a key enzyme in the synthesis of the active form of Vitamin D₃ (1α,25-(OH)₂D₃), would have increased blood pressure relative to wild-type (WT) littermates.

Knock-out (KO) and WT mice were instrumented with telemetry transmitters; baseline blood pressure and heart rate were continuously recorded and reported as 24-hour means for 7 days. In a subset of animals (n=4/group) heart and kidney were isolated for mRNA quantification for the vitamin D receptor (VDR), 25-hydroxyvitamin D-24-hydroxylase (CYP24A1), renin, and natriuretic peptide precursor A (NPPA) by real-time RT-PCR. Throughout the 7 days of telemetry recordings, mean arterial pressure (MAP) was significantly elevated in KO mice (24-hour group means between 112±2 and 116±4 mmHg; n=7) relative to WT littermates (between 106±2 and 108±2 mmHg; n=10). Additionally, KO mice were tachycardic throughout the same period (24-hour group means between 601±6 and 610±7 beats/min) relative to WT controls (between 544±6 and 566±4 beats/min). Heart to body weight and LV to body weight ratios also trended up for KO vs. WT mice reflecting an increased load on the cardiac muscle. Concomitant with the increase in blood pressure in KO mice, renal renin mRNA expression trended up (n=4) relative to WT littermates while VDR mRNA expression decreased. NPPA was modestly elevated in the hearts of KO animals likely reflecting a compensatory effect in response to hypertension and hypertrophy.

The above results demonstrate that disruption of the CYP27B1 gene product, effectively leading to 1α,25-(OH)₂D₃ deficiency, produces a sustained elevation in blood pressure and heart rate relative to 1-alpha hydroxylase-replete animals. These results also suggest that the increases in MAP may be mediated by a deregulation of the renin-angiotensin system.

### EXAMPLE 7

### Differential Effects of Vitamin D Analogs on Vascular Calcification in Uremic Rats

Vitamin D receptor activators (VDRAs) are commonly used to manage secondary hyperparathyroidism associated with chronic kidney disease (CKD). Recent clinical data show that VDRAs provide survival benefit for stage 5 CKD. In CKD patients, vascular calcification is often linked to an unfavorable prognosis. In this study, the calcium and phosphorus content in aorta isolated from uremic rats was measured. ⁴⁵Ca uptake into cultured aorta rings was also examined.

The 5/6 nephrectomized rats were obtained from Charles River Labs. (Wilmington, Mass.). Two weeks after nephrectomy, rats were put on a diet containing 0.9% phosphorous and 0.6% calcium for 4 weeks, followed by treatment with vehicle (5% ethanol+95% propylene glycol, 0.4 ml/kg), paricalcitol or doxercalciferol at 0.67 μg/kg, i.p., 3 times/week, for 2 weeks. Twenty-four hours after the last dosing, blood was collected via tail vein under ketamine anesthesia (50 mg/kg) and aorta collected for calcification studies. The combination of 5/6 nephrectomy and high phosphorus diet resulted in hypocalcemia (serum calcium: 1.06 vs. 1.28 mmol/L in naive animal), hyperphosphatemia (11.6 vs. 7.5 mg/dL) and an elevation in serum PTH (17.3-fold), creatinine (2.9-fold) and BUN (3.8-fold). After the aortas were removed in a sterile manner from the rats, the aventitia was gently removed and each aorta was washed three times with medium. A segment of each aorta was processed for calcium and phoshphorus determination. A separate portion of each aorta was denuded, cut into 2-3-mm rings and placed in DMEM containing 0.2 μCi/ml ⁴⁵Ca for 3 days at 37° C. Afterwards, aortic rings were washed, dried, weighed, and then dissolved to determine radioactivity. Results show that: (1) there was a linear correlation between calcium and phosphorus content and ⁴⁵Ca uptake in the aorta, (2) there was a modest (1.6-fold) increase in Ca content, but no significant difference in ⁴⁵Ca uptake or phosphorus content in the paricalcitol-treated group when compared to vehicle, and (3) ⁴⁵Ca uptake was 40-fold higher, Ca content 1.0-fold higher and phosphorus content 17-fold higher in the doxercalciferol-treated group when compared to the vehicle group. Both paricalcitol and doxercalciferol at 0.67 μg/kg elevated iCa (˜25%) and suppressed PTH (>90%). These results suggest that doxercalciferol and paricalcitol exhibit different effects on aorta calcification in uremic rats.

### EXAMPLE 8

### Effect of Vitamin D Analogs on Calcification in Human Vascular Smooth Muscle Cells

Chronic kidney disease (CKD) patients experience a high mortality rate from cardiovascular diseases. Vitamin D receptor activators (VDRAs) such as paricalcitol, doxercalciferol and calcitriol are commonly used to manage secondary hyperparathyroidism associated with CKD. Recent clinical data show that VDRAs provide survival benefit for Stage 5 CKD patients. In CKD patients, vascular calcification is often linked to an unfavorable prognosis. Previous studies have shown that 1,25(OH)₂D₃ (calcitriol) at 10⁻⁷ to 10⁻⁹ M induced a dose dependent increase in calcification of bovine vascular smooth muscle cells (SMC) in vitro (Jono et al., 2000). In this study, the effect of Vitamin D analogs on ⁴⁵Ca uptake into primary culture of human coronary artery sooth muscle cells (CASMC) was examined.

Primary cultured human CASMC were grown to >80% confluence and used within five passages. Cells were treated with drugs for 5 days in the appropriate medium, and then changed back to base medium (N1: DMEM containing 1.8 mM Ca⁺² and 0.9 mM PO₋₄) containing 0.2 μCi/ml ⁴⁵Ca for 24 hr. Afterwards, cells were washed with PBS three times and radioactivity measured by liquid scintillation. Cells cultured in N1 medium exhibited minimal ⁴⁵Ca uptake (98±14, n=5). Cells treated with inducing medium (P1-high phosphorus: DMEM containing 1.8 mM Ca⁺², 3.8 mM PO₋₄ and 7.5 U/ml alkaline phosphatase) exhibited a dramatic increase in ⁴⁵Ca uptake. The effect of the inducing medium was dose-dependent; a mixture of 90% N1/10% P1 or 60% N1/40% P1 increased ⁴⁵Ca uptake by 7.4 and 34.4-fold, respectively. Paricalcitol at 100 nM did not show a significant effect on ⁴⁵Ca uptake in cells treated with N1 medium or different doses of P1 medium (20-60%). As a control, paricalcitol stimulated CYP24A1 and suppressed PAI-1 mRNA expression in a dose-dependent manner. When cells in N1 or 60% N1/40% P1 media were treated with increasing concentrations of paricalcitol, calcitriol or activated doxercalciferol (1-100 nM), no significant effects on ⁴⁵Ca uptake from the drugs were observed. These results suggest that human CASMC cultured in medium containing high phosphorus plus alkaline phosphatase exhibit increased calcium uptake, and Vitamin D analogs have no significant effect.

### EXAMPLE 9

### Differential Effects of Paricalcitol and Doxercalciferol on Serum PTH and Ionized Calcium in Uremic Rats With Established Secondary Hyperparathyroidism

It is clinically important to suppress elevated serum PTH levels in ESRD patients with established secondary hyperparathyroidism (SHPT) independent of hypercalcemia. Moreover, the 5/6 uremic rat model has effectively predicted the clinical profile of vitamin D analogues with respect to PTH and hypercalcemia. Thus, a direct comparison was conducted of paricalcitol (PARI) and doxercalciferol (DOX) in male Sprague Dawley rats subjected to 5/6 nephrectomy (surgical ablation) placed on 4 weeks of a high phosphorous diet (0.9% phos., 0.6% Ca) to establish SHPT. On Day 0, rats received vehicle (VEH; 5% ethanol, 95% propylene glycol; 0.4 ml/kg; IP) or drug (0.083, 0.17, 0.33, 0.66 mcg/kg; n=7-10/group) 3 times/week for 12 days. Blood samples were collected 24 hr post dose (Day 13). Day 0 values (mean±SEM) for: creatinine (VEH vs sham) were 1.02±0.05 vs 0.48±0.01 mg/dL; PTH, 1320±185 vs 178±11 pg/ml; blood ionized calcium (iCa; 1.26±0.01 vs 1.30±0.01 mmol/L); phosphorous (8.70±0.34 vs 7.36±0.09 mg/dL). In VEH, the ratio of Day 13/Day 0 iCa values declined to 0.93±0.02 vs 1.02±0.01 for sham; PTH and phosphorous tended to increase. In contrast, DOX dose-dependently increased Day 13/Day 0 Ca++ ratios to 1.09±0.03* and 1.17±0.04* at 0.33 and 0.66 mcg/kg doses. PARI had no effect on iCa at 0.33 mcg/kg whereas the iCa ratio increased modestly to 1.05±0.02*# at 0.66 mcg/kg, less than that of DOX. The Day 13/Day 0 PTH ratios for DOX fell to 0.43±0.12, 0.26±0.06*, and 0.22±0.11* in response to 0.18, 0.33, 0.66 mcg/kg, respectively, while PARI decreased PTH ratios to 0.37±0.07*, 0.33±0.06*, 0.13±0.02*. Serum phosphorus was not affected in any treatment group. DOX produced a dose-dependent increase in active calcium transport in duodenum ex vivo; PARI had no effect. Thus, in the uremic rat with established SHPT, DOX suppresses serum PTH levels concomitant with dose-dependent elevations in ionized calcium greater than those caused by equal doses of. PARI. At these doses, PARI effectively reduces PTH without eliciting dose-dependent changes in ionized blood calcium.
*=p<0.05 vs. VEH ANOVA; # p<0.05 unpaired t test DOX vs PARI

### EXAMPLE 10

### Inhibition of Renin Biosynthesis by the Vitamin D Analog Paricalcitol

The renin-angiotensin system (RAS) plays an essential role in the regulation of electrolyte and volume homeostasis. Over-activation of the RAS is associated with high blood pressure and other cardiovascular and renal diseases such as cardiac hypertrophy and diabetic nephropathy. Renin is the first and rate-limiting enzyme of the renin-angiotensin cascade and thus represents an important therapeutic target; however, renin inhibitors are not currently available. It has been shown that 1,25-dihydroxyvitamin D₃ [1,25(OH)₂D₃], the hormonal form of vitamin D, is an endocrine suppressor of renin biosynthesis, which provides a molecular basis to explore the potential of vitamin D analogs as renin inhibitors to control the RAS.

In the present study, an investigation was made of the in vitro and in vivo activity of paricalcitol, a FDA-approved low calcemic vitamin D analog, to inhibit renin biosynthesis. To this end, As4.1-hVDR cells, a JG cell-like cell line stably transfected with human VDR, was treated with paricalcitol or 1,25(OH)₂D₃ at doses ranging from 10⁻¹⁰ to 10⁻⁷ M for 24 hours, and its renin-inhibiting activity was assessed by Northern blot analyses; the cells were also transfected with renin gene promoter-luciferase reporter plasmid, and luciferase activity was determined in the presence and absence of paricalcitol or 1,25(OH)₂D₃. These in vitro studies demonstrated that paricalcitol suppresses renin mRNA expression and renin gene transcription in a dose-dependent manner, with a potency comparable to that of 1,25(OH)₂D₃.

To assess the in vivo activity of this compound, CD-1 male mice (n=7) were treated with paricalcitol by intraperitoneal injection at the doses of 1.5 and 3.0 μg/kg body weight, 3 times a week for three weeks, and the changes in body weight, blood ionized calcium, renal renin mRNA and plasma renin activity (PRA) were assessed at the end of the treatment. The data showed that paricalcitol at these two doses had no effect on body weight and only slightly increased the levels of blood ionized calcium; however, paricalcitol at these two doses significantly reduced the levels of both renal renin mRNA (by 23% to 45%) and PRA (by 20% to 70%) in the treated mice. These data establish, in principle, that paricalcitol can indeed inhibit renin biosynthesis and suggest that paricalcitol may potentially be used to control renin production.

### EXAMPLE 11

### Effect of Paricalcitol on Renin mRNA Expression in Vitamin D-Deficient Rats

Chronic kidney disease (CKD) patients encounter a much higher risk of cardiovascular disease than the general public. Several vitamin D receptor activators including paricalcitol are currently available for the treatment of hyperparathyroidism secondary to CKD. Recent clinical data show that paricalcitol provides survival benefit for CKD patients. Previously, it has been shown that vitamin D receptor activators suppress the expression of renin in AS4.1 cells. To investigate whether paricalcitol at non-calcemic doses would regulate renin in vivo, renin mRNA expression was examined in the kidney of vitamin D deficient rats treated with paricalcitol.

Weanling Sprague-Dawley male rats housed in an environment free of UV light were fed a 0.4% Ca, 0.3% P, vitamin D-deficient diet for 5 weeks, then switched to a 0.02% Ca, 0.5% P, vitamin D-deficient diet for 21 days. Rats were dosed with 0.003, 0.01, 0.03, and 0.3 μg/kg of paricalcitol, i.p., three times per week, for the final 2 weeks. A control group of animals was given vehicle only (5% ethanol/95% propylene glycol), and untreated, age-matched animals on normal diet (0.5% Ca, 0.4% P) served as normal peer control. Twenty-four hours after the last dose, the rats were anesthetized with ketamine (100 mg/kg), cardiac bled, killed by CO₂, tissues collected for RNA extraction and real-time RT-PCR analysis, and serum taken for determination of calcium, phosphorus, and PTH. The diet treatment resulted in hypocalcemia (ionized calcium 0.70±0.02 vs. 1.34±0.01 mmol/L in normal peer control), elevated serum PTH (29-fold) and phosphorus (9.3±0.3 vs. 8.1±0.2 mg/dl), and an increase in renal renin mRNA (+20%). Paricalcitol at tested doses did not result in a significant change in calcium, but reduced serum PTH dose-dependently. Paricalcitol treatment normalized renal renin mRNA expression to levels found in normal, vitamin D-sufficient animals.

The above data show that, in vitamin D-deficient rats with elevated PTH and hypocalcemia, paricalcitol at doses that do not affect serum calcium significantly suppresses renal renin mRNA expression. The effect of paricalcitol on suppressing renin expression may be a factor contributing to reduced mortality and morbidity risk in paricalcitol-treated CKD patients.

We claim:

1. A sustained release pharmaceutical composition for preventing, treating and delaying progression of cardiovascular, cerebrovascular and peripheral vascular diseases, especially heart failure, cardiomyopathy, atherosclerosis, myocardial infarction, and cerebrovascular accident, comprising:
a therapeutically effective amount of a VDRA or Vitamin D analog; and optionally a therapeutically effective amount of at least one member of the group consisting of an angiotensin converting enzyme inhibitor, an angiotensin (II) receptor (I) blocker, and an aldosterone blocker.

2. A sustained release pharmaceutical composition according to claim 1, wherein said VDRA or Vitamin D analog is selected from the group consisting paricalcitol, calcitriol and doxercalciferol.

3. A sustained release pharmaceutical composition according to claim 1, wherein said composition is in the form of a transdermal patch.

4. A sustained release pharmaceutical composition according to claim 1, wherein said composition is in an oral dosage form.

5. A sustained release pharmaceutical composition according to claim 1, wherein said composition is in a subcutaneous dosage form.

6. A sustained release pharmaceutical composition according to claim 1, wherein said composition is in an injectable dosage form.

7. A sustained release pharmaceutical composition according to claim 6, wherein said injectable dosage form is a member of the group consisting of a subcutaneous dosage form and a depot dosage form.

8. A sustained release pharmaceutical composition according to claim 5, wherein said composition is in an implantable form.

9. A pharmaceutical composition for treating, preventing or delaying progression of vascular disease in a mammal, comprising:
a therapeutically effective amount of Vitamin D receptor activator or Vitamin D analog; and optionally a therapeutically effective amount of at least one member of the group consisting of an angiotensin converting enzyme inhibitor, an angiotensin (II) receptor (I) blocker, and an aldosterone blocker.

10. The pharmaceutical composition according to claim 9, wherein said cardiovascular disease is selected from the group consisting of heart failure, cardiomyopathy, atherosclerosis, myocardial infarction, cerebrovascular accident and peripheral vascular disease.

11. The pharmaceutical composition according to claim 9, wherein said Vitamin D receptor activator or Vitamin D analog is selected from the group consisting of paricalcitol, calcitriol, and doxercalciferol.

12. The pharmaceutical composition according to claim 9, wherein said composition is in transdermal patch form.

13. The pharmaceutical composition according to claim 9, wherein said composition is in oral dosage form.

14. The pharmaceutical composition according to claim 9, wherein said composition is in subcutaneous dosage form.

15. The pharmaceutical composition according to claim 9, wherein said composition is in an injectable dosage form.

16. The pharmaceutical composition according to claim 15, wherein said injectable dosage form is a member selected from the group consisting of a subcutaneous dosage form and a depot dosage form.

17. The pharmaceutical composition according to claim 14, wherein said composition is in an implantable form.

18. A method of preventing, treating and delaying disease progression of vascular disease in a mammal, comprising the step of administering to said mammal a pharmaceutical composition according to claim 9.

19. The method according to claim 18, wherein the administering step is continuous.

20. The method according to claim 18, wherein the administering step is carried out using a transdermal patch.

21. The method according to claim 18, wherein the administering step is carried out using an oral dosage form.

22. The method according to claim 18, wherein the administering step is carried out using an injectable dosage form.

23. The method according to claim 18, wherein the administering step is carried out using a subcutaneous dosage form.

24. A method of treating, inhibiting or preventing vascular disease in a mammal by reducing PAI-1 expression in said mammal, comprising the step of administering to said mammal an effective amount of a Vitamin D receptor activator or Vitamin D analog.

25. The method according to claim 24, wherein said Vitamin D receptor activator is paricalcitol or calcitriol.

26. The method according to claim 24, wherein said Vitamin D analog is doxercalciferol or alfacalcidol.

27. A method of treating, inhibiting or preventing thrombosis in a mammal in need of said treatment, inhibition or prevention, comprising the step of administering to said mammal an effective amount of a Vitamin D receptor activator or Vitamin D analog.

28. The method according to claim 27, wherein said Vitamin D receptor. activator is paricalcitol or calcitriol.

29. The method according to claim 27, wherein said Vitamin D analog is doxercalciferol or alfacalcidol.
